(19) United States
(12) Patent Application Publication
(10) Pub. No.: US 2015/0141741 A1
(43) Pub. Date: May 21, 2015

(54) THERAPEUTIC ISOLATION TANK

(71) Applicant: SAN JUAN PATENTS, INC., Lakeland, FL (US)

(72) Inventor: KIRK SULLIVAN, LAKELAND, FL (US)

(73) Assignee: SAN JUAN PATENTS, INC., Lakeland, FL (US)

(21) Appl. No.: 14/083,700

(22) Filed: Nov. 19, 2013

(51) Int. Cl. A61M 21/00; A61H 33/02; A61H 33/00

(52) U.S. Cl. CPC A61M 21/0094; A61H 33/00; A61H 33/02; A61H 33/6005; A61H 2033/048
USPC 600/26; 29/428

(57) ABSTRACT

A float tank for use in therapeutic floatation therapy. The float tank is specifically configured and adapted to be lightweight and formed in sections to allow a single individual to transport and install the float tank in both indoor and outdoor locations. The float tank may also include a pump which generates microbubbles within an Epsom Salt solution used for floatation therapy which may enhance the therapeutic effect achieved during a floatation therapy session.

### CROSS-REFERENCE TO RELATED APPLICATIONS

Not Applicable

### STATEMENT RE: FEDERALLY SPONSORED RESEARCH/DEVELOPMENT

Not Applicable

### BACKGROUND

1. Technical Field

The present disclosure generally relates to a therapeutic isolation tank, and more specifically to a tank configured for floatation therapy in an oxygenated water solution, wherein the tank is adapted to be easily installed in a desired location.

2. Related Art

Sensory deprivation therapy is known in the art for achieving many physical and mental benefits. In general, sensory deprivation therapy includes the deliberate reduction or removal of stimuli from one or more senses. Although simple devices, such as blindfolds, hoods and earmuffs may reduce one's sight and/or hearing, there is a desire to interrupt the senses of smell, touch, taste, thermoception (i.e., heat-sense) and “gravity.”

One particular technique which achieves higher levels of sensory deprivation includes floatation therapy, wherein an individual floats in a concentrated solution of Epsom Salts having a specific gravity which allows the individual to float in a relaxed position with their face above surface of the solution. The solution is preferably maintained at a temperature wherein the user feels neither hot nor cold. Such floatation therapy sessions are typically performed in a closed environment, wherein the individual is shielded from external sounds or light. The absence of gravity and externally generated stimuli generally allows the individual to obtain extremely deep rest and relaxation. The floatation therapy may also allow the individual to achieve healing from injury or illness in a manner not readily achievable by other non-drug based means.

In view of the significant physical and mental benefits readily attributable to floatation therapy, specialized chambers have been developed specifically for floatation therapy. Such floatation chambers are typically built-in to an existing residence or therapeutic facility (i.e., a spa) as an additional room within structure. The floatation chambers are specifically constructed in a manner which isolates the interior chamber from the external environment, and thus, the walls surrounding the chamber may be lined with specialized sound and/or light insulation. Furthermore, the floatation chambers also generally include plumbing for heating and filtering the solution used for the floatation therapy. As such, the plumbing is typically routed through the walls of the existing structure.

Although such built-in floatation chambers tend to be effective in conducting floatation therapy, the construction of such built-in floatation chambers tends to be a timely, complicated, and costly endeavor. In particular, it may be difficult to find a location within an existing residence or therapeutic facility to build a new floatation chamber. Furthermore, even if the space exists, there may be difficulty with integrating the plumbing and electrical needs of the floatation chamber with the existing plumbing and electrical lines of the existing structure. Moreover, it may take a team of construction workers several days, or weeks to complete construction of the built-in float tank, which generally makes such built-in float tanks very costly and available to only wealthy individuals.

Accordingly, there is a need in the art for an improved float tank which is specifically configured and adapted to allow for easy transportation and installation of the float tank, either in a location inside an existing structure, or alternatively, in an outdoor location. There is also a need in the art for a float tank which enhances the therapeutic effect achieved during floatation therapy. Various aspects of the present invention are directed toward addressing these needs, as will be discussed in more detail below.

### BRIEF SUMMARY

According to an aspect of the invention, there is provided a float tank for use in therapeutic floatation therapy. The float tank is specifically configured and adapted to be lightweight and formed in sections to allow a single individual to transport and install the float tank in both indoor and outdoor locations. The float tank may also include a hydrotherapy generator which generates microbubbles (e.g., very small bubbles of oxygen) within the solution used for floatation therapy which may enhance the therapeutic effect achieved during a floatation therapy session.

According to one embodiment, there is provided a float tank configured to allow for human floatation in a liquid medium. The float tank includes a base having a plurality of support elements and a peripheral wall circumscribing the plurality of support elements. A tub is positionable on the base, with the tub defining a tub reservoir sized and configured to receive a quantity of the liquid medium and to allow a human user to lie within the tub reservoir. The support elements are configured to support a load applied by the tub when the tub is positioned on the base. The float tank further includes a sidewall member having a tub end portion and an opposed roof end portion. The tub end portion is positionable over the tub and the opposed roof end portion extends away from the tub. A roof is positionable on the sidewall member and extends over the tub reservoir. The tub, sidewall member and roof collectively define an internal chamber. Furthermore, the roof and sidewall member collectively define an opening sized and configured to allow the human user to enter/leave the internal chamber.

The float tank may further include a door moveably coupled to the sidewall member and roof and moveable relative thereto between a closed position and an open position, wherein the door substantially covers the opening when the door is in the closed position. The door may create a substantially fluid tight seal with sidewall member and roof when the door is in the closed position.

The sidewall member may include a pair of opposed longitudinal sidewalls, and a pair of opposed end walls extending between the pair of opposed longitudinal sidewalls in spaced relation to each other. At least one of the pair of opposed end walls may form an acute angle with corresponding edges of the pair of opposed longitudinal sidewalls. The opening may be formed in the at least one of the pair of opposed end walls forming an acute angle with corresponding edges of the pair of opposed longitudinal sidewalls. The roof may include an end wall sized and configured to be flush with the pair of opposed end walls.

The float tank may additionally include a hydrotherapy generator having an outlet in fluid communication with the tub reservoir and configured to emit gas into the tub reservoir.

The base, tub, sidewall member and roof are configured to be separable from each other to facilitate movement of the float tank. The tub, sidewall member and roof may be fabricated from a fiberglass material.

According to another aspect of the invention, there is provided a method of installing the float tank, wherein the float tank is provided in several discrete sections, including a lower member, an intermediate member and an upper member. When assembled, the lower, intermediate and upper members collectively define an internal float chamber. The method further includes placing the lower member on a support surface, and then placing the intermediate member on the lower member. The method further includes placing the upper member on the intermediate member.

The presently contemplated embodiments will be best understood by reference to the following detailed description when read in conjunction with the accompanying drawings.

### BRIEF DESCRIPTION OF THE DRAWINGS

These and other features and advantages of the various embodiments disclosed herein will be better understood with respect to the following description and drawings, in which:

FIG. 1 is an upper perspective view of a therapeutic isolation float tank constructed in accordance with an embodiment of the present invention;

FIG. 2 is a front view of the therapeutic isolation float tank having a door in an open position;

FIG. 3 is an exploded upper perspective view of the therapeutic isolation float tank depicted in FIG. 1;

FIG. 4 is a side sectional view of the float tank with a human user lying in the float tank and depicted in phantom; and

FIG. 5 is a schematic view of electrical and fluid components of the float tank.

Common reference numerals are used throughout the drawings and the detailed description to indicate the same elements.

### DETAILED DESCRIPTION

Referring now to the drawings, wherein the showings are for purposes of illustrating a preferred embodiment of the present invention only and not for purposes of limiting the same, there is depicted a therapeutic isolation float tank 10 constructed in accordance with a preferred embodiment of the present invention. The float tank 10 is specifically configured and adapted for easy transport, assembly and operation. Along these lines, the float tank 10 is divided into several discrete component sections which allow the float tank 10 to be carried and installed by a single user (in sections) in an existing structure, such as a residential home. Furthermore, the electrical components used in connection with the float tank 10 are preferably adapted to receive power from a standard household circuit. In this regard, various aspects of the float tank 10 are directed toward allowing the float tank 10 to be installed and used at a user's residence, either inside the residence or at a location outside the residence, such as on a porch, deck, or the like.

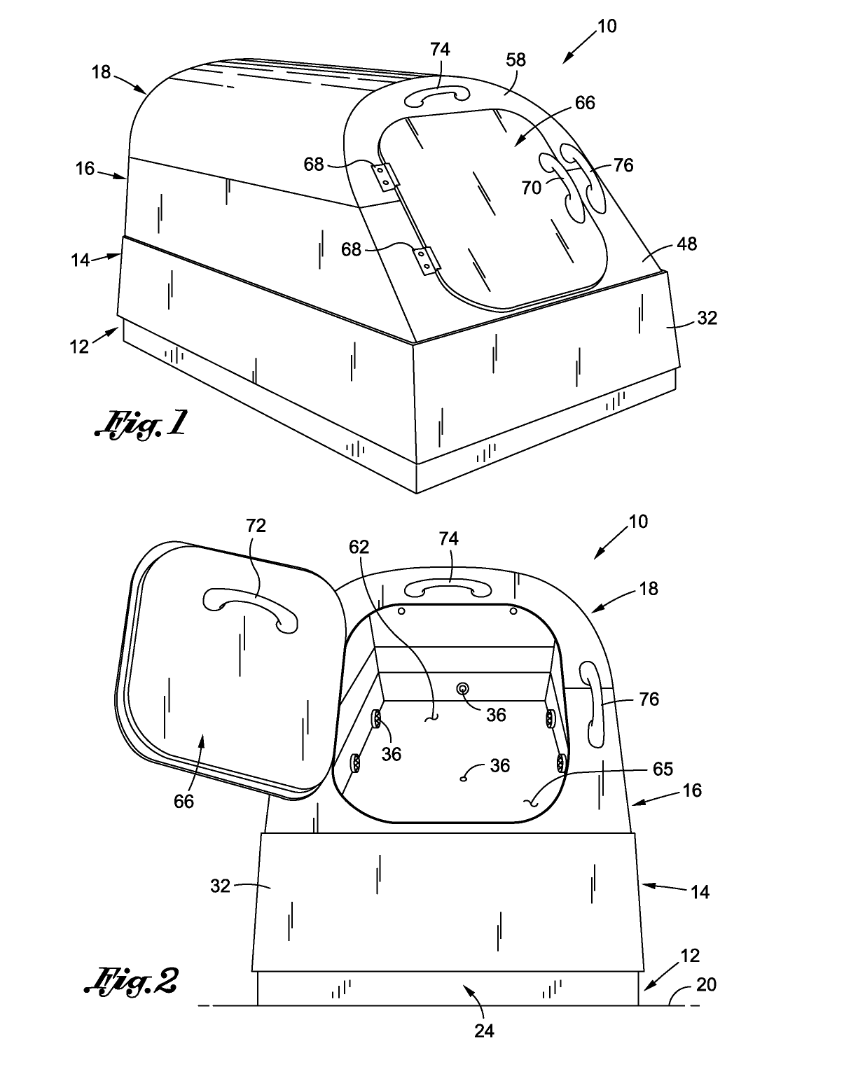

Referring now specifically to FIG. 1, the exemplary float tank 10 is divided into several sections/components which generally includes a base 12, a tub 14, a sidewall member 16 and a roof 18. Those components are configured to be stacked/assembled upon each other in the manner shown in the drawings to construct the float tank 10. The components are preferably formed from durable, yet lightweight material, such as fiberglass, although other materials known in the art may also be used.

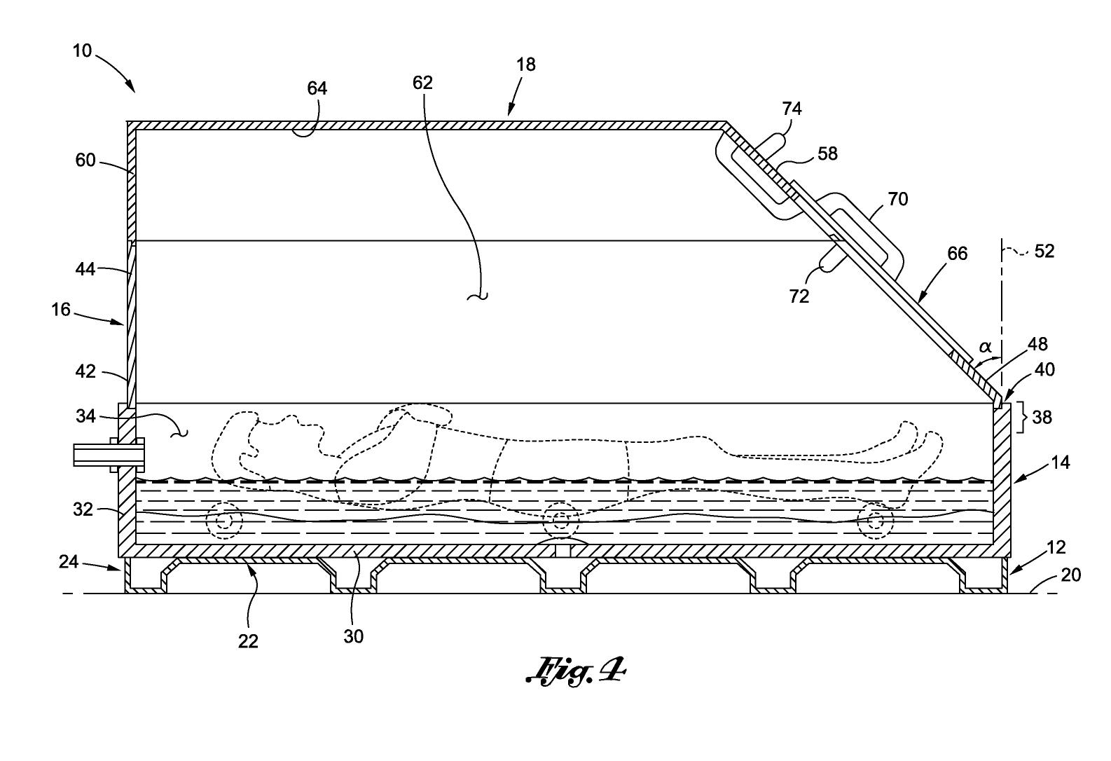

The bottom of the float tank 10 includes the base 12, which is disposable on an underlying support surface 20, such as the ground, flooring, a deck, etc. The base 12 generally includes a plurality of support elements 22 and a peripheral wall 24 circumscribing the plurality of support elements 22. In the exemplary embodiment shown in FIG. 3, the base 12 includes eight support elements 22 arranged in a 4×2 array, although the number and arrangement of support elements 22 may vary without departing from the spirit and scope of the present invention.

The base 12 defines a generally rectangular configuration, wherein the peripheral wall 24 includes a pair of opposed longitudinal wall sections and a pair of opposed latitudinal wall sections extending between the pair of longitudinal wall sections in spaced relation to each other. The peripheral 24 wall also terminates at an upper edge 26, which resides in a plane that is substantially parallel to the underlying support surface 20.

The support elements 12 are configured to support a load applied by the tub 14, as will be described in more detail below. To that end, each support element 22 includes a planar engagement face 28 configured to engage with or bear the load of the tub 14. The plurality of planar engagement faces 28 are positioned within a substantially common plane to provide a level support. Preferably, the plane containing the plurality of planar engagement faces 28 also includes the upper edge 26 of the peripheral wall 24. As an alternative, the upper edge 26 of the peripheral wall 24 may be slightly recesses so as not to bear the load applied by the tub 14.

The tub 14 is positionable over the base 12, with a bottom wall 30 of the tub 14 resting on top of and being supported by the plurality of support elements 22. Extending upwardly from the tub bottom wall 30 is a tub sidewall 32 which defines a tub reservoir 34 collectively with the bottom wall 30. The tub reservoir 34 is sized and configured to receive a quantity of the liquid medium used during the floatation therapy. A common liquid used during floatation therapy is water having Epsom Salt dissolved therein, although other liquids may also be used without departing from the spirit and scope of the present invention. The tub reservoir 34 is further sized and configured to allow a human user to lie therein.

According to one embodiment, the tub 14 is approximately 45″ wide, 84″ long and 12″ deep, although it is understood that the dimensions of the tub 14 may vary without departing from the spirit and scope of the present invention.

The tub sidewall 32 and bottom wall 30 include one or more plumbing openings 36 formed therein. As will be described in more detail below, the plumbing openings 36 may be used as a drain, water return, hydrotherapy port, etc.

The upper end portion 38 of the tub sidewall 32 is configured to support the tank sidewall member 16, which rests on top of the tub sidewall 32. According to one embodiment, the upper end portion 38 of the tub sidewall includes a shoulder 40 that is sided and configured to support the tank sidewall member 16.

The sidewall member 16 is sized and configured to increase the height of the float tank 10. The sidewall member 16 includes a bottom end portion 42 (i.e., a tub end portion), and an opposed upper end portion 44 (i.e., a roof end portion). The bottom end portion 42 terminates to define a lower edge which rests on and is supported by the shoulder 40 of the tub sidewall 32. In this regard, the size and shape of the lower edge is substantially complimentary to the size and shape of the tub sidewall shoulder 40.

According to one embodiment, the sidewall member 16 includes a pair of opposed longitudinal sidewalls 46, as well as a forward end wall 48, and an opposing rear end wall 50 extending between the pair of opposed longitudinal sidewalls 46 in spaced relation to each other. The forward end wall 48 is preferably angularly offset from a vertical plane by an angle α so as to form an acute angle with corresponding bottom edges of the pair of opposed longitudinal sidewalls 46. In one implementation of the invention, the angle α is substantially equal to 45°, although the magnitude of the angle α may vary without departing from the spirit and scope of the present invention.

A roof 18 is positionable on the sidewall member 16 and extends over the tub reservoir 34. In the exemplary embodiment, the roof 18 defines a curved upper portion and an opposing lower end portion including a lower edge 54 which interfaces with an upper end portion 44 of the sidewall member 16. The roof 18 includes a forward roof end wall 58 and an opposing rear roof end wall 60. When the roof 18 is placed on the sidewall member 16, the forward end wall 58 of the roof 18 is substantially flush with the forward end wall 48 of the sidewall member 16. In this regard, the forward end wall 58 of the roof 18 is also preferably angularly offset from the vertical plane 52 by an offset angle α.

When the float tank 10 is assembled, the tub 14, sidewall member 16, and the roof 18 collectively define an internal float chamber 62. The internal float chamber 62 is large enough for a human user to comfortably and easily maneuver himself/herself therein.

Ingress and egress into and out of the float chamber 62 is achieved via an opening 65 collectively defined by the roof 18 and sidewall member 16. The opening 65 is preferably formed in the angled forward end walls 58, 48 of the roof 18 and sidewall member 16. The angled orientation of the end walls 58, 48 allow the user lying within the internal float chamber 62 to look upwardly through the opening 65 to the outside environment. For instance, depending on the placement of the float tank 10, the user may look up to the night sky, overhanging foliage, passing clouds, or other soothing and relaxing scenes.

According to another embodiment, the inner surface 64 of the roof 18 may include an image depicted thereon. Along these lines, if the individual floating in the tank 10 does not want to completely deprive himself/herself of light, the inner surface 64 of the roof 18 may include a relaxing scene/image depicted thereon. For instance, a picture of the earth viewed from outer space may be created/imprinted on the inner surface 64 of the roof 18.

Although the floatation tank 10 may be primarily used with the intention of isolating as many external stimuli as possible, it is also understood that some individuals may want to experience floatation without complete sensory deprivation. Along these lines, the float tank 10 may include one or more elements which stimulate the individual's sense(s) in a relaxing manner. For instance, the float tank may include fiber optic or LED lighting to create soothing ambient lighting or speakers to create soothing ambient sounds. The lighting and/or speakers may be mounted on the internal surface of the float tank 10.

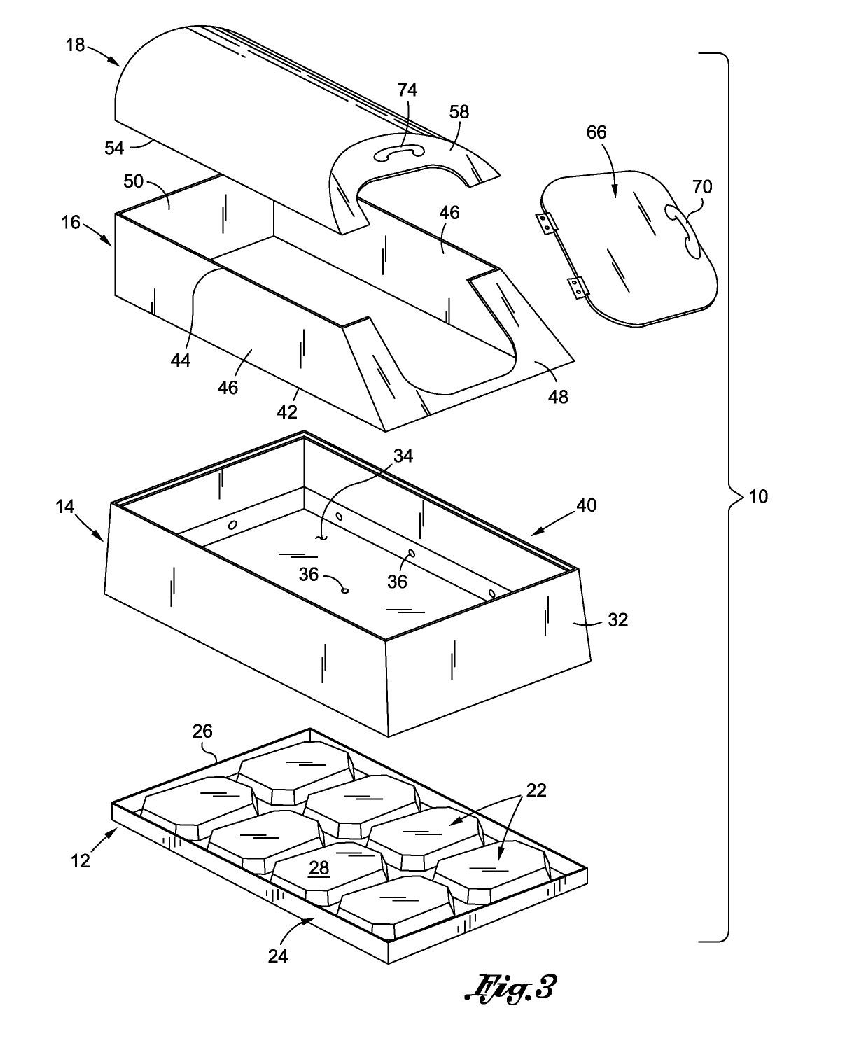

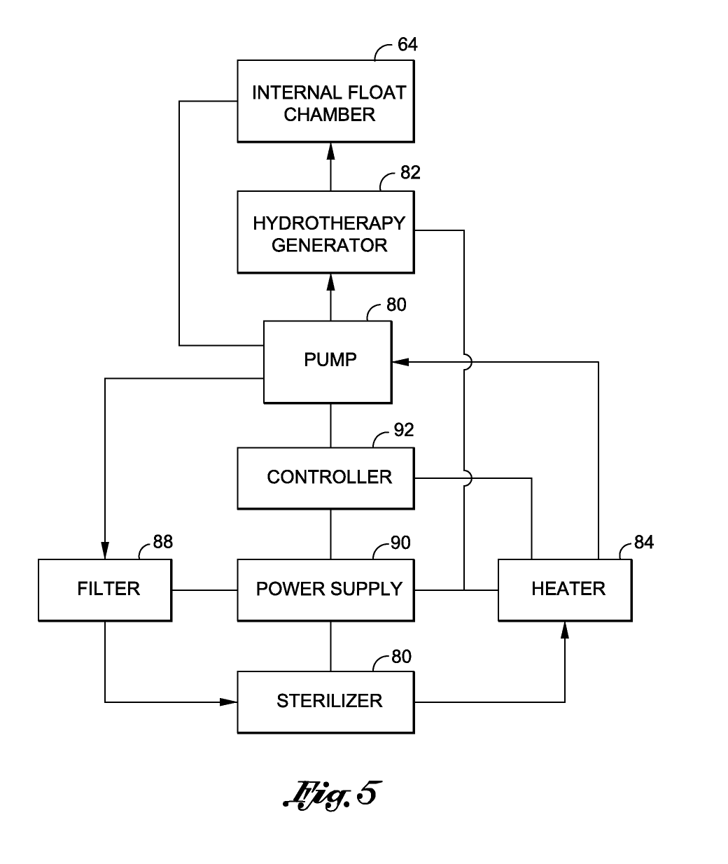

The float tank 10 further includes a door 66 moveably coupled to the sidewall member 16 and roof 18 and moveable relative thereto between a closed position (see FIGS. 1 and 4) and an open position (see FIG. 2). According to one embodiment, the door 66 is hingedly connected to the sidewall member 16 and roof 18 via a pair of pivoting hinges 68. When the door 66 is in the closed position, the door 66 substantially covers the opening 65. The door 66 is moved from the closed position toward the open position to allow for entry and exit into and out of the internal float chamber 62. The hinge(s) 68 may be “friction hinges” which allow the hinge(s) 68 to define a limited range of motion to stop the door 66 in the open position. In other words, the hinge(s) 68 may limit pivotal movement of the door 66 to less than 180° to allow a user located within the internal float chamber 68 to more easily reach the door 66 for closing the door 66. The door 66 may substantially restrict light from entering the internal chamber 62 when the door 66 is in the closed position.

Although the exemplary embodiment includes a pivoting door 66, it is also contemplated that other embodiments may include a door which translates, rotates or otherwise moves between the closed and open positions.

The float tank 10 may include one or more handles to allow a user to open the door 66 and to provide a gripping point when entering or exiting the float tank 10. In the exemplary embodiment, the float tank 10 includes an external door handle 70 mounted on the outside of the door 70 and an internal door handle 72 mounted on the inside of the door 66. Furthermore, the tank 10 includes first and second external gripping handles 74, 76 mounted to the exterior surface of the float tank 10, and more specifically to the roof 18 and sidewall member 16.

Referring now specifically to FIG. 5, there is depicted a schematic view of various fluid and electrical components which are used during operation of the float tank 10. In particular, the float tank 10 includes a pump 80, hydrotherapy generator 82, a heater 84, a sterilizer 86 and a filter 88. The pump 80 is configured to circulate the Epsom Salt solution through the fluid system, which includes the internal float chamber 64, and the various fluid components. Preferably, the pump 80 is a salt water pump capable of pumping the water at 9 gallons/minute.

The hydrotherapy generator 82 is preferably configured to generate and emit oxygenated water including a plurality of microbubbles which are able to enter the skin's pores, remove impurities, carry oxygen for improved metabolism, lightly exfoliate the skin and improve hydration for dry skin. The term “microbubble” is used herein to refer to a bubble having a diameter of less than approximately 50 microns. An exemplary hydrotherapy pump 82 is manufactured by JASON INTERNATIONAL, based in North Little Rock, Ark., in connection with their MICROS ILK products, although other microbubble generators known by those skilled in the art may also be used.

In order to maximize the floatation therapy experience, the water/Epsom Salt solution is preferably pumped through the internal chamber 62 at a comfortable temperature. As such, the water heater 84 preferably heats the water to a temperature that is neither hot nor cold to the touch, although a user may control the temperature of the water to any desired temperature. In one implementation of the present invention, the water heater is a 13-amp electric titanium heater.

The sterilizer 86 is fluidly coupled to the pump 80 and sterilizes the water to maintain the cleanliness of the water. According to one embodiment, the sterilizer 86 is a high output DELTA UV sterilizer. A filter 88 may also be used in connection with the sterilizer 86 for further water filtration. The filter 88 is preferably a 40 square foot filter, although other filter sizes may be used without departing from the spirit and scope of the present invention.

In order to simply operation of the float tank 10, the pump 80, filter 88, sterilizer 86, hydrotherapy generator 82 and heater 84 are configured to plug into a standard household power outlet 90. As such, complicated re-wiring or connections with the existing electrical system of the structure is not required. As an alternative, the float tank 10 may include a dedicated power source, such as a battery or generator for supplying power to the system.

The float tank 10 may additionally include a controller 92 with a user interface which allows a user to control operation of the float tank 10. In this regard, the user may use the controller 92 to turn the float tank 10 ON or OFF, as well as to set the temperature of the water, control the hydrotherapeutic effects, and any entertainment options (i.e., lighting, speakers, etc). The controller 92 may additionally be capable of operating the float tank in accordance with one or more pre-set operational modes with assigned operational parameters. For instance, a pre-set mode may automatically operate the float tank 10 for a prescribed duration, with the water at a prescribed temperature, and perform a prescribed hydrotherapy. The pre-set modes may be pre-programmed by the manufacturer or may be set by the user(s) after purchase.

With the basic structure of the float tank 10 described above, the following discussion will describe a typical installation of the float tank 10. As noted above, the float tank 10 is formed in separate and discrete sections to allow the float tank 10 to be carried into an existing structure one piece at a time, and then subsequently assembly in the structure. In this regard, the various sections are preferably sized and configured to allow each section to be carried into the structure without removing doors or windows (i.e., the sections are preferably configured to fit through a door that is 2.5 feet wide or larger). It is also contemplated that the float tank 10 may be assembled in an outdoor location, such as on a porch or deck using the same method described herein in relation to an indoor installation.

The float tank 10 is preferably installed on a substantially flat, horizontal surface 20. According to one embodiment, the footprint of the tank 10 is approximately 52×91 inches, and thus, enough space should be cleared before bringing the sections to the installation site. The base 12 is placed on the surface 20 in the orientation shown in FIG. 3 (i.e., with the planar engagement faces 28 facing upwardly). The tub 14 is then placed on the base 12, and then the sidewall member 16 on top of the tub 14. The roof 18 is then placed on top of the sidewall member 16.

The door 66 is then connected to the assembled structure such that the door 66 covers the opening 65 defined by the roof 18 and sidewall member 16 when the door 66 is in the closed position. The door 66 may be coupled to the structure by securing the hinges 68 to the forward faces 48, 58 of the sidewall member 16 and roof 18. Any handles fastened to two adjacent sections (i.e., such as the roof and sidewall member), may also be attached at this time.

When the primary structure is assembled, the fluid and electrical components may be connected thereto. In this regard, the drains and returns in the internal chamber may be connected to the pump to allow for fluid circulation through the system. Furthermore, the electrical components are then connected to the power supply 90, such as a standard household circuit.

After the float tank 10 has been set up, the float tank 10 is ready for use. According to one embodiment, the float tank 10 is filled with approximately 100 gallons of water with approximately 900 lbs. of Epsom Salt dissolved therein. This combination of water and Epsom Salt creates approximately a 1.3 specific gravity fluid which typically allows a user's body to be lifted and supported evenly on all sides.

The user may set the operational parameters of the system using the controller 92, as described above. When the float tank 10 is ready (i.e., when the temperature of the water has reached the level set by the user), the user can step into the floatation chamber 62 by opening the door 66. If the user wants to leave the door 66 open, the friction hinges 68 may maintain the door 66 in the open position to give the user the option of looking upwardly through the opening 65. Conversely, if the user desires darkness, the door 66 can be closed.

Once inside the float tank 10, the user may lay in the Epsom Salt solution to begin the floatation session. The floatation session may last for as long as the user desires (e.g., 30 minutes to 3 hours). When used for purposes of sensory deprivation, the user tends to experience no sensation of hot or cold, and instead the water solution is merely a warm blanketing effect that removes sensation from the user's skin. When the user opens his/her eyes, there is no stimulation of light. Furthermore, with the user's ears underwater and with earplugs in, the user finds that the external stimulations cease, which allows the user to experience new levels of relaxation and chiropractic stretching.

The benefits associated with floatation therapy achievable when using the float tank 10 include reduced chronic pain, promotion of creative thinking, strengthened immune system, a calming of the body and mind, decreased stress and an improvement in one's sense of well-being.

In addition to the foregoing, there are several benefits associated with using microbubble hydrotherapy during a floatation session. During a typical float session, billions of oxygen microbubbles are introduced into the float chamber 64 to refresh the user's body, rejuvenate the user's skin, and bring total relaxation. According to various aspects of the invention, the microbubbles increase the oxygen level in the water up to 70% more than common tap water. The microbubbles are small and negatively charged, which allows the microbubbles to enter the user's skin pores and remove toxins, while also providing oxygen and anions to the epidermis and upper dermis layers of the skin.

Furthermore, floating in Epsom Salt has been found to promote the absorption of Magnesium and Sulphates, which many consider to be vital to good health and well being.

This disclosure provides exemplary embodiments of the present invention. The scope of the present invention is not limited by these exemplary embodiments. Numerous variations, whether explicitly provided for by the specification or implied by the specification, such as variations in structure, dimension, type of material and manufacturing process may be implemented by one of skill in the art in view of this disclosure.

What is claimed is:

1. A float tank configured to allow for human floatation in a liquid medium, the float tank comprising:
a base having a plurality of support elements and a peripheral wall circumscribing the plurality of support elements;
a tub positionable on the base, the tub defining a tub reservoir sized and configured to receive a quantity of the liquid medium and to allow a human user to lie within the tub reservoir, the support elements being configured to support a load applied by the tub when the tub is positioned on the base;
a sidewall member having a tub end portion and an opposed roof end portion, the tub end portion being positionable over the tub and the opposed roof end portion extending away from the tub; and
a roof positionable on the sidewall member and extending over the tub reservoir;
the tub, sidewall member and roof collectively defining an internal chamber;
the roof and sidewall member collectively defining an opening sized and configured to allow the human user to enter/leave the internal chamber.

2. The float tank recited in claim 1, further comprising a door moveably coupled to the sidewall member and roof and moveable relative thereto between a closed position and an open position, the door substantially covering the opening in the closed position.

3. The float tank recited in claim 2, wherein the door creates a substantially fluid tight seal with sidewall member and roof when the door is in the closed position.

4. The float tank recited in claim 1, wherein the sidewall member includes a pair of opposed longitudinal sidewalls, and a pair of opposed end walls extending between the pair of opposed longitudinal sidewalls in spaced relation to each other, at least one of the pair of opposed end walls forming an acute angle with corresponding edges of the pair of opposed longitudinal sidewalls.

5. The float tank recited in claim 4, wherein the opening is formed in the at least one of the pair of opposed end walls forming an acute angle with corresponding edges of the pair of opposed longitudinal sidewalls.

6. The float tank recited in claim 4, wherein the roof includes an end wall sized and configured to be flush with the pair of opposed end walls.

7. The float tank recited in claim 1, further comprising a hydrotherapy generator having an outlet in fluid communication with the tub reservoir and configured to emit a gas into the tub reservoir.

8. The float tank recited in claim 7, wherein the hydrotherapy generator emits gas bubbles having a diameter of substantially 50 microns or less.

9. The float tank recited in claim 1, wherein the base, tub, sidewall member and roof are configured to be separable from each other to facilitate movement of the float tank.

10. The float tank recited in claim 1, wherein the tub, sidewall member and roof are fabricated from a fiberglass material.

11. A float tank configured to allow for human floatation in a liquid medium, the float tank comprising:
a lower member having a plurality of support elements and a peripheral wall circumscribing the plurality of support elements;
an intermediate member positionable on the lower member, the intermediate member defining a reservoir sized and configured to receive a quantity of the liquid medium and to allow a human user to lie within the reservoir, the support elements being configured to support a load applied by the intermediate member when the intermediate member is positioned on the lower member; and
an upper member being positionable over the intermediate member and extending over the reservoir;
the lower member, intermediate member and upper member collectively defining an internal chamber;
the upper member defining an opening sized and configured to allow the human user to enter/leave the internal chamber.

12. The float tank recited in claim 11, further comprising a door moveably coupled to the upper member and moveable relative thereto between a closed position and an open position, the door substantially covering the opening in the closed position.

13. The float tank recited in claim 12, wherein the door creates a substantially fluid tight seal with upper member and when the door is in the closed position.

14. The float tank recited in claim 11, wherein the upper member includes a pair of opposed longitudinal sidewalls, and a pair of opposed end walls extending between the pair of opposed longitudinal sidewalls in spaced relation to each other, at least one of the pair of opposed end walls forming an acute angle with corresponding edges of the pair of opposed longitudinal sidewalls.

15. The float tank recited in claim 14, wherein the opening is formed in the at least one of the pair of opposed end walls forming an acute angle with corresponding edges of the pair of opposed longitudinal sidewalls.

16. The float tank recited in claim 11, further comprising a hydrotherapy generator having an outlet in fluid communication with the reservoir and configured to emit gas into the reservoir.

17. The float tank recited in claim 16, wherein the hydrotherapy generator emits gas bubbles having a diameter of substantially 50 microns or less.

18. The float tank recited in claim 11, wherein the lower member, intermediate member and upper member are fabricated from a fiberglass material.

19. A method of installing a float tank configured to allow for human floatation in a liquid medium, the method comprising the steps of:
providing a float tank including:
a lower member having a plurality of support elements and a peripheral wall circumscribing the plurality of support elements;
an intermediate member positionable on the lower member, the intermediate member defining a reservoir sized and configured to receive a quantity of the liquid medium and to allow a human user to lie within the reservoir, the support elements being configured to support a load applied by the intermediate member when the intermediate member is positioned on the lower member; and
an upper member being positionable over the intermediate member and extending over the reservoir;
the lower member, intermediate member and upper member collectively defining an internal chamber;
the upper member defining an opening sized and configured to allow the human user to enter/leave the internal chamber;
placing the lower member on a support surface;
placing the intermediate member on the lower member; and
placing the upper member on the intermediate member.

20. The method recited in claim 19, wherein the providing step includes providing a float tank includes including a hydrotherapy generator configured to emit gas bubbles into the liquid medium having a diameter of substantially 50 microns or less.
